(19) United States
(12) Patent Application Publication
(10) Pub. No.: US 2019/0216345 A1
(43) Pub. Date: Jul. 18, 2019

(54) METHOD AND SYSTEM FOR MONITORING AND DISPLAYING PHYSIOLOGICAL CONDITIONS

(71) Applicant: Christoper Scheib, Nicholasville, KY (US)

(72) Inventor: Christoper Scheib, Nicholasville, KY (US)

(21) Appl. No.: 16/164,560

(22) Filed: Oct. 18, 2018

Related U.S. Application Data
(60) Provisional application No. 62/573,840, filed on Oct. 18, 2017.

(51) Int. Cl. A61B 5/048; A61B 5/04; A61B 5/00

(52) U.S. Cl. CPC A61B 5/048; A61B 5/04012; A61B 5/165; A61B 5/4064; A61B 5/4821

(57) ABSTRACT

Evaluating Electroencephalogram (EEG) data includes receiving initial EEG signals for a patient being subjected to an anesthetic agent associated with a first probability for responding to surgical stimulation; identifying at least one expected pathway of EEG signals, wherein the at least one expected pathway of EEG signals comprises a time-ordered series of expected EEG signals for the patient based on the anesthetic agent; receiving a subsequent EEG signal for the patient after a change to a level of the anesthetic agent has occurred; based on the change to the level of the anesthetic agent, determining a change from the first probability to a second probability for responding to surgical stimulation; and calculating an amount of increase to an analgesic agent to administer to the patient to return substantially to the first probability for responding to surgical stimulation.

### RELATED APPLICATIONS

The present application claims priority to U.S. Provisional Patent Application Ser. No. 62/573,840, filed Oct. 18, 2017, the disclosure of which is incorporated herein in its entirety.

### BACKGROUND

The present invention relates generally to monitoring brain function during different states of consciousness such as general anesthesia, coma or natural sleep and, more particularly, to using electroencephalogram (EEG) data and other physiological data to evaluate brain function.

The definition of the term “anesthesia”—is—a lack of awareness—or lack of sensation. For surgical purposes this is generally achieved in two main ways; 1) infiltration of a peripheral or more central nerve bundle with a local anesthesia, which prevents the nerve impulse being processed by the central nervous system and, thus, sensation (of pain or otherwise is not perceived by the individual who remains conscious and aware; and 2) general anesthesia which requires a loss of consciousness in order for the sensation not to be perceived by the individual. To date no systems of monitoring brain function has produced a reference point beyond which one can absolutely state that there exists a complete lack of consciousness at an anesthetic dosage level low enough to be of practical value. Present systems merely produce a measure of probability of loss of consciousness when the anesthetic dosage level is at the low end of the practical range.

The “depth of anesthesia” generally describes the extent to which consciousness is lost following administration of an anesthetic agent. As the magnitude of anesthetization, or depth of anesthesia, increases, an anesthetized patient typically fans to successively respond to spoken commands, loses the eyelid reflex, loses other reflexes, undergoes depression of vital signs, and the like. Once consciousness is lost there is a progression of effects on brain function as higher concentrations or dose of anesthetic agent are administered.

For clinical use, it is desirable to simplify the results of EEG signal analysis of the foregoing, and other types, into a workable parameter that can be used by an anesthesiologist in a clinical setting when attending the patient. Prior techniques have included slimming the EEG signal in a relatively unprocessed form or showing a number (or letter) without any other underlying data supporting that number. Neither solution is helpful in a clinical setting; especially, in the case of the “number” indicator, when the number is at best a probability that the patient is not aware or conscious.

The present application is related to U.S. Pat. No. 9,770,205 filed Oct. 14, 2014 and U.S. patent application Ser. No. 15/797,221 filed Oct. 30, 2017 which claims priority and benefit of, U.S. patent application Ser. No. 14/513,803 filed Oct. 14, 2014, which claims priority to U.S. Provisional Patent Application Ser. No. 61/889,578, filed Oct. 11, 2013 and is a continuation-in-part of the previously filed U.S. patent application Ser. Nos. 12/925,295 filed Oct. 18, 2010 and 12/925,296 filed Oct. 18, 2010 (now U.S. Pat. No. 8,401,631), which themselves are continuation-in-part applications of the previously filed U.S. patent application Ser. No. 12/589,047 filed Oct. 16, 2009 (now U.S. Pat. No. 8,352,021) which is a continuation-in-part of the previously filed U.S. patent application Ser. No. 12/082,842 filed Apr. 15, 2008 (now U.S. Pat. No. 7,720,531), the disclosures of which are incorporated herein in their entirety.

### SUMMARY

Embodiments of the present invention relate to a method for Evaluating Electroencephalogram (EEG) data includes receiving initial EEG signals for a patient being subjected to an anesthetic agent associated with a first probability for responding to surgical stimulation; identifying at least one expected pathway of EEG signals, wherein the at least one expected pathway of EEG signals comprises a time-ordered series of expected EEG signals for the patient based on the anesthetic agent; receiving a subsequent EEG signal for the patient after a change to a level of the anesthetic agent has occurred; based on the change to the level of the anesthetic agent, determining a change from the first probability to a second probability for responding to surgical stimulation; and calculating an amount of increase to an analgesic agent to administer to the patient to return substantially to the first probability for responding to surgical stimulation.

It is understood that other embodiments of the present invention will become readily apparent to those skilled in the art from the following detailed description, wherein it is shown and described only various embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWINGS

Various aspects of a system and method for anesthesia monitoring are illustrated by way of example, and not by way of limitation, in the accompanying drawings, wherein:

FIG. 1: EEG Spectra on a log-log graph with arrows pointing to an alpha peak at 11 Hertz and a theta trough at 7 Hertz.

FIG. 2: Multiple EEG spectra superimposed on the same log-log graph. The x axis is frequency in Hertz. The legend indicates the time during the procedure. Sevoflurane was used with decreasing concentration as time progressed.

FIG. 3: The thin line spectrum illustrates the point in the pathway of the maximum alpha peak and the thick line spectrum is at a higher concentration. The segment to the left of the peak is the low frequency segment and the segment to the right of the peak is the high frequency segment. At the higher concentration the peak is smaller, the low frequency segment is steeper and the high frequency segment is less steep.

FIG. 4: The thin line spectrum illustrates the point in the pathway of the maximum alpha peak and the thick line spectrum is at a lower concentration. As the estimated effect site concentration decreased the alpha peak became smaller and power at all frequencies below the alpha peak decreased. The power at frequencies above the alpha peak did not change.

FIG. 5: The thick line EEG spectrum is the same as the thick line in FIG. 4. Further reduction of the estimated effect site concentration results in loss of power at all frequencies.

FIG. 6: The EEG spectral pathways from fifteen patients anesthetized with sevoflurane were evaluated and examples of the maximum alpha peak spectra were selected. The patients ranged in age across five decades. The thick line is the average of the fifteen spectra. The spectra from a 23 year old and a 56 year old are also shown.

FIG. 7: The powers of two of the EEG spectra in FIG. 6 were adjusted. The spectrum of the 23 year old was decreased by 6 decibels and the spectrum of the 56 year old was increased by 4 decibels.

FIG. 8A: Two spectra from a case with halogenated inhaled agent. The thick line spectrum illustrates the point in the pathway of the maximum alpha peak and the thin line spectrum is at a higher concentration.

FIG. 8B: Two spectra from the same case as FIG. 8A. The thick line spectrum illustrates the point in the pathway of the maximum alpha peak and the thin line spectrum is at a lower concentration.

FIG. 9: Two of the most important features from the EEG spectra that were used in FIGS. 3, 4, and 5 are the power at the center frequency of the alpha peak and the lowest point of the theta trough. Tracking these two parameters against the calculated effect site concentration of propofol shows the concentration range for the zone of the maximum alpha peak power. This figure also shows how the two parameters change at concentrations above and below that zone.

FIGS. 10A-D: These figures illustrate four different pathways.

FIG. 11: The solid line EEG spectrum is from a patient at 1.35% isoflurane before incision. The dashed line is the EEG spectrum after incision and at the same concentration of isoflurane.

FIG. 12: illustrates that increasing the isoflurane concentration to 1.61% isoflurane shifted the spectrum back to being almost identical to the spectrum at 1.35% isoflurane before incision.

FIG. 13: Reducing the sevoflurane concentration produced multiple changes in the EEG spectrum.

FIG. 14: Adding an additional dose of fentanyl without changing the sevoflurane concentration largely reversed those changes.

FIG. 15: Superimposing the EEG spectra of 2% sevoflurane and 1% sevoflurane plus additional fentanyl illustrates how much the additional fentanyl reversed the changes.

FIG. 16: The curved line indicates the relationship between the calculated effect site concentration of fentanyl and the amount of halogenated agent (or propofol) to produce an adequate anesthetic. The line represents a value that is the average for a group.

FIG. 17: The curved line from FIG. 16 can be separated into three zones according to the rate of increase in the amount of opioid required as the level of halogenated agent or propofol is reduced.

FIG. 18: Changes in the EEC spectra with reductions of halogenated agent (or propofol) and the effect of increasing the level of opioid can be used to determine a subject's location on the curved line of FIG. 16. A is 3% sevoflurane. B is 2% sevoflurane, C is 1% sevoflurane. D is 1% sevoflurane plus additional fentanyl.

FIG. 19: The parameters of alpha peak center frequency power and theta trough or peak power can be used to locate a patient on the halogenated agent (and propofol) versus opioid requirement curve,

FIG. 20: The Desflurane effect site concentration was reduced from 10% in steps to 3.8% and then increased. The alpha peak height is the difference in decibels between the power at the center frequency of the alpha peak and the theta trough or peak. The numerous dips were due to responses to surgery and sometimes noise from the use of electrocautery.

FIG. 21: The spectra are from the same case as FIG. 20. The effect site Desflurane concentration is 3.8% for all three spectra. The changes are due to the effect of surgical stimulation.

FIGS. 22A-D: The spectra show a paradoxical response. There are power decreases in alpha and theta and a power increase in delta (0.25-3.5 Hertz) as a response to surgical stimulation. The amount of the power changes increased as the Desflurane concentration was reduced to 5.3%. This concentration produced the largest power changes. Smaller power changes occurred with a further reduction to 4.8%.

FIG. 22: This illustrates paradoxical responses where the heart rate goes up and the spectral edge frequency (SEF) goes down. The paradoxical responses are largest at 5.3% (FIG. 220).

### DETAILED DESCRIPTION OF THE PREFERRED EMBODIMENTS

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

Embodiments in accordance with the principles of the present disclosure encompass a system and method that determines from a series of EEG signals and other patient data the approximate probability of a subject responding to surgical stimulation prior to surgical stimulation and the effectiveness of the anesthetic, state with regard to blocking responses to surgical stimulation after it has occurred. The system and method also determines from a series of EEG signals and other patient data an estimation of the change in probability of a subject responding to surgical stimulation if the primary (inhaled halogenated anesthetic agent or propofol) anesthetic and the analgesic agent levels change. The information provided enables the anesthesia provider to maintain an adequate anesthetic as the primary agent is decreased by having a reasonable estimation of the amount of opioid analgesic required with each reduction of the primary agent.

Over the years many papers have been published which reviewed neuroscience topics and suggested that they may be important to understanding anesthesia. But so far, few clinicians have used this information to change the way they practice. EEG based monitors were developed empirically and are alleged to measure the “depth” of anesthesia which is a clinical, not a neuroscience based concept. It is debatable whether or not these monitors have changed anesthesia practice.

There is enough literature to support the concept that an EEG signal can indicate that a patient is not conscious. This can be seen in a variety of presentations including the density spectral array (DSA) which is available as a display option on many commercial EEG monitors. However, the DSA can indicate the patient is not conscious well below the level of one minimum alveolar concentration (MAC). This means that even though a pattern in the EEG indicates that a patient is not conscious, it does not mean that they will not move or otherwise respond to surgical stimulation. At a medical meeting for clinicians a professor of anesthesia was explaining how the DSA indicated that the patient was not aware and clinician participants said that level of agent in that example was not adequate to control blood pressure or prevent movement. One participant asked, “Why not just use the calculated MAC value from the end-tidal agent analysis to guide your anesthetic.” Neuroscience based monitoring must address the issues of how much analgesia would be needed and how effective is the current level of analgesia if it is likely to gain widespread clinical use. The clinical advantage of a neuroscience based monitor that indicates a patient is unconscious is the sensitivity or resistance to anesthetic agents of individual patients can be determined and the level of anesthetic, agent can be reduced below the levels commonly used without the benefit of such a monitor. This can only be done routinely if the clinician knows how much analgesia is needed to prevent response to surgical stimulation as the inhaled agent or propofol is reduced.

When the end-tidal concentration of a halogenated agent is increased from about 0.7 to 1.5 MAC the EEG spectrum changes in a consistent manner for most patients. The power in the trough of the theta range continues to rise after the power of the peak in the alpha frequency range reaches a maximum. By the definition of MAC it is clear that at 1.5 MAC very few patients would require opioid supplementation. From 1.2 MAC and lower it would we advisable to maintain a constant level of opioid analgesia that would need to be increased as the MAC level is reduced further. The relationship between a) the theta trough or peak relative to the alpha peak changing with anesthetic agent concentration and b) the changing requirement for opioid supplementation may not be coincidental and might be explained using a neuroscience based explanation. Propofol does not produce the large increase in theta amplitude that halogenated inhalation agents do. Also propofol is not very effective without supplemental analgesia at blocking movement responses to surgical stimulation. Propofol does not have as much of an effect on potassium channels either. Perhaps the power of the theta trough or peak rises with increasing inhalation agent because of effects on potassium channels results in greater hyperpolarization than the maximum effect on GABA channels. The reversal potential for potassium ions is much more negative than the reversal potential for chloride ions which enter the neuron when GABA channels open. Perhaps the lack of movement response that is the basis of MAC is due to effects on potassium channels in motor neurons causing hyperpolarization beyond the level of hyperpolarization that can occur with GABA effects. This degree of hyperpolarization may be sufficient to prevent action potentials even with the maximum depolarizing synaptic input. If the degree of hyperpolarization is reduced from there a movement response will occur unless analgesic effects reduce the sensory related input to the motor neuron. These two proposed mechanisms could indirectly link theta power to opioid requirement.

Even if the EEG could accurately predict opioid requirement, a method to indicate the effectiveness of analgesia would be beneficial to minimize sudden responses to surgical stimulus. Warnings before an impending response would be beneficial for clinicians to fully utilize a system that enabled the use of lower than routine levels of anesthetic agent. As anesthetic agent and opioid level decrease toward a response to surgical stimulation the relationship may be a sigmoid curve. If this is true one would want to run the anesthetic in the plateau at the top and want to have indications if the patient is dose to the “edge” or beginning to move down the slope. When the EEG spectrum during anesthesia has a peak in the alpha range, there are at least two shape features that occur in the frequency ranges higher than the peak. With a log-log presentation there is often a straight line sloping steeply to the upper end of the spectrum. There can also be a “bump” or peak in the beta frequency range. The presence or absence of this beta activity may be an indication of opioid effectiveness.

An example of an EEG guided protocol would be to track the changing shape and power of the EEG spectrum with special attention to the theta and alpha regions. As the primary agent is reduced and power in the theta region decreases the level of opioid analgesia would be maintained or increased. If a “beta bump” occurred in the EEG spectrum, additional doses of opioid would be administered in an effort to eliminate it. Other indications of surgical stimulation affecting the EEG would also be watched for, Cardiovascular responses to surgical stimulation without EEG changes might be better treated with cardiovascular agents than anesthetic agents. EMG responses would be interpreted as a failure of analgesia.

Implementing this protocol would benefit from a display that can superimpose two individual spectra. This would clearly show relative changes in the theta and alpha regions as well as a “beta bump” before it becomes a beta peak. A DSA would not likely do this very well. Controlling the level of analgesia would utilize pharmacokinetic model with the EEG analysis display.

In summary, implementing neuroscience based anesthesia practice uses methods of EEG analysis and monitor displays that address the requirement and effectiveness of analgesia at lower levels of propofol and inhaled agents. The description below explains the theoretical basis for how this can be achieved and why its implementation would produce a better anesthetic.

One of the most commonly observed oscillations in the EEG signal during anesthesia is in the alpha frequency range (7-14 Hz). This occurs in the front half of the brain and is different than the alpha oscillations which occur in the occipital (back) region in subjects when they are awake. Two detailed mechanisms have been proposed to explain frontal alpha oscillations during anesthesia. They are the spindle oscillations produced during natural sleep and the traveling peak mechanism.

It is known that natural sleep produces spindle waves which are EEG oscillations that occur approximately within the alpha range. The experimental preparations that were used to develop the mechanism of sleep spindles most often involved some form of anesthesia. The spindle and traveling peak mechanisms as they apply to anesthetized subjects are of interest. The sleep spindle mechanism applied to anesthesia will be referred to herein as the spindle mechanism.

Cortical pyramidal, thalamocortical, and reticular thalamic neurons are three types of neurons that are required to produce spindle waves that are detectable in the EEG. Reticular neurons are GABAergic, mutually inhibit, and hence can synchronize each other. Since reticular neurons project to thalamocortical neurons, reticular neurons can synchronize thalamocortical neurons together with other reticular thalamic neurons. Thalamocortical neurons are excitatory and project to both pyramidal and reticular neurons. Some cortical pyramidal neurons project back to thalamocortical and reticular thalamic neurons exciting both and thus completing a loop. If the membrane potential of reticular and thalamocortical neurons are in a narrow range, hyperpolarized a few millivolts from their usual potential when awake, spindle waves may occur. In this membrane potential range, depolarizing input from corticothalamic or thalamocortical neurons will cause reticular thalamic neurons to depolarize and start cycles of depolarization and hyperpolarization at the frequency of spindle waves. In the depolarizing phase these neurons will fire bursts of action potentials. These action potentials will release GABA which will hyperpolarize thalamocortical neurons. The result is a rebound depolarization which may end with an action potential. This action potential produces dendrite potentials in pyramidal neurons at the frequency of spindle waves. The EEG primarily reflects dendrite potentials in pyramidal neurons since these neurons are oriented parallel to each other and perpendicular to the surface of the cortex. Reticular thalamic neurons determine the frequency of the spindle waves and can be loosely understood to be pacemakers for spindle waves.

The widely acknowledged concept that the membrane potential of both reticular thalamic and thalamocortical neurons must be in a narrow range of membrane potential for spindle oscillations to occur is related to a calcium channel (T channels) that both types of neurons possess. The T channel requires a period of hyperpolarization prior to depolarization in order for it to open. T channels can remain open and depolarize the neuron for a much longer period of time than depolarization produced as a result of a sodium channel based action potential. This prolonged depolarization can result in multiple action potentials instead of a single action potential. This is referred to as a “burst” and that the thalamocortical and reticular thalamic neurons have transitioned into “burst mode”.

The main points of this sleep spindle mechanism are that the frequency of the oscillation is determined largely by the reticular nucleus of the thalamus and does not produce oscillations substantially above or below the alpha range. All that is required to produce spindles is that thalamocortical and reticular thalamic neurons are in a particular range of membrane potential that is more negative than the membrane potential of these neurons when a subject is awake. This situation occurs during natural sleep when the brainstem projection of depolarizing neuromodulators such as acetylcholine, histamine, and norepinephrine are reduced from levels that occur in subjects who are awake.

Anesthesia could change the membrane potential by any of several known effects. Possible mechanisms include potentiating GABA receptors, potentiating potassium ion channels, and/or inhibiting lh ion channels in reticular thalamic and thalamocortical neurons. The above mentioned mechanisms for change in membrane potential would be considered to be direct effects on thalamic neurons. Indirect mechanisms would include mimicking sleep by decreasing depolarizing projections from the brainstem to the thalamus.

There are differences between the characteristics of sleep spindles and anesthesia induced alpha oscillations. The major difference is that sleep spindles usually consist of sets of approximately a dozen oscillation cycles which are temporally separated from each other by gaps of several seconds. Alpha oscillations observed during anesthesia can have many more cycles in a set and the sets can occur less than one second apart. These differences may be explained by anesthetic agent inhibition of the lh channel. It is thought that hyperpolarization during sleep spindles activates lh which depolarizes thalamic neurons and terminates the spindle. During natural sleep this prevents another spindle for a few seconds. Models of sleep spindles and anesthesia induced alpha rhythms indicate that inhibition of lh would result in continuous spindles.

Theories of consciousness based on synchronization of 40 Hz oscillations have been proposed previously. It is not unreasonable that 40 Hz oscillations are major components of the mechanism of consciousness but such theories remain incomplete. The traveling peak mechanism involves anesthetic agent effects on GABA receptors reducing the frequency of a 40 Hz oscillation that is generated in the cortex.

A study conducted with volunteers to whom propofol was administered In a stepwise manner and that tracked induction and emergence found that during sedation prior to loss of consciousness a peak in the EEG spectrum occurred in the beta frequency range (13-24 Hz). The frequency of this peak decreased as the level of propofol increased and entered the alpha frequency range (8-12 Hz) at loss of consciousness. During emergence and the return of consciousness the frequency of the observed peak increased back into the beta range. The peak in the EEG spectra was named the “traveling peak” because it changed frequency with propofol level. Emergence was the reverse of induction in regard to the frequency shift in this traveling peak. Beta band activity with sedation and anesthesia induction has been reported before and has been established as a common phenomenon.

A neurophysiologic mechanism for the traveling peak was proposed and tested with a computer model. The model and proposed mechanism involve a 40 Hz oscillation which occurs in cortical neurons prior to anesthesia. GABAergic inhibitory interneurons are critical to the cortical generation of the 40 Hz oscillation. As the level of propofol increases in the brain. GABA is potentiated at an increasing rate prolonging its inhibitory effects, which results in a decrease in the frequency of the oscillation. It has been proposed that when the frequency decreases to the alpha range the cortical based oscillation entrains the thalamic neurons into the oscillation. The mechanism of consciousness continues to function but with continuously increasing impairment up until this point when the thalamus is entrained by the cortical rhythm. The entrainment of thalamic, neurons includes the neurons of the reticular nucleus of the thalamus. Reticular thalamic neurons then synchronize with many thalamocortical neurons which in turn synchronize large areas of the cortex through the thalamocortical loop. The synchronization of large cortical areas is not compatible with consciousness. In theory, at that point the subject would not be able to process sensory input or stored memories at the level of the frontal cortex. This proposed mechanism will be referred to herein as the “traveling peak mechanism”.

In a later article this proposed mechanism was expanded to include a second mechanism to explain the loss of occipital alpha rhythms. These rhythms are well known and occur only in the awake state with eyes dosed and the subject relaxed. A third mechanism was proposed for the occurrence of frontal alpha rhythms which occur after the loss of consciousness with anesthesia as the occipital alpha oscillations end. The phenomenon of alpha rhythms stopping in the occipital cortex and beginning in the frontal cortex at loss of consciousness is also well known. This observation has been termed “anteriorization of alpha”. Since occipital alpha rhythms end and frontal alpha rhythms begin approximate to the loss of consciousness, it may appear that the same mechanism causes both with just a shift in location. However, in reality they are two different mechanisms.

The second and third mechanisms cited the effect of propofol on both GABA receptors and the voltage regulated ion channel, hyperpolarization-activated cation current “lh” in thalamic neurons. The inhibition of lh by propofol has been established. The lh channel is a nonspecific cation channel that predominantly increases sodium conductance. Hyperpolarization activates this channel which then depolarizes neurons shortening the duration of hyperpolarization induced by other ion channels. Anesthetic agents inhibit this channel causing thalamic, neurons to remain longer at a hyperpolarized membrane potential. The model proposes that this action of anesthetic agents stops occipital cortex projecting thalamic neurons from producing action potentials in the alpha frequency range. Those neurons have ion channels which produce oscillations in the alpha range when depolarized and do not produce these oscillations when hyperpolarized. In contrast, frontal cortex projecting thalamic neurons have a different set of voltage dependent ion channels. Hyperpolarization caused by the inhibition of lh will allow these thalamocortical and reticular thalamic neurons to activate each other in a “ping pong” manner. The frequency of this “ping pong” effect is in the alpha range because of the kinetics of the hyperpolarization activated voltage gated ion channels present in these neurons. The cortical traveling peak mechanism entrains thalamic neurons into their oscillation when the frequency decreases into the alpha range. Added to this concept of entrainment of a cortical based oscillation can be added an alpha oscillation intrinsic to the thalamus due to the “ping pong” effect between reticular thalamic and thalamocortical neurons.

A computer model indicates that if the lh channel is completely inhibited the alpha oscillations will not stop. The “ping pong” mechanism is similar to the mechanism of sleep spindles and may be considered a variation of the mechanisms proposed for sleep spindles. One major difference between the “ping pong” and the classic sleep spindle mechanism is that in the “ping pong” model the oscillation is created by thalamocortical and reticular thalamic neurons stimulating each other in a reciprocal fashion. The sleep spindle mechanism proposed by M. Steriade emphasized that the reticular thalamic neurons paced the oscillation because a surgically isolated reticular nucleus continued to produce the characteristic oscillation. In subsequent publications by Steriade and colleagues, the mechanism allowed that there were significant effects on the oscillation by the interaction between cortical, thalamocortical, and reticular thalamic, neurons.

Natural sleep produces its characteristic EEG phenomena as a result of a reduction of brainstem induced depolarization of the thalamus and cortex. There are multiple brainstem nuclei which project depolarizing neuromodulators to the thalamus and cerebral cortex. There is a nucleus in the brainstem that can affect the activity of all of these nuclei. This nucleus is the ventrolateral preoptic (VLPO) nucleus. When the VLPO is active (“on”) it projects GABA to multiple brainstem nuclei which reduce their projection of depolarizing neuromodulators to the thalamus and cortex. When the VLPO is less active (“off”) these brainstem nuclei are released from inhibition. These same nuclei also project back to the VLPO which acts to shut down the VLPO when the arousal nuclei are active. This relationship has been called a “flip flop switch” because there are sudden changes between on and off. There can be confusion since when the VLPO is “on” the brainstem arousal system is “off” due to active inhibition. If the VLPO is “off” the brainstem arousal system is released from active inhibition. To avoid confusion the phrase brainstem arousal system is “on” (or “off”) will be used instead of the VLPO is “off” or “on”.

The VLPO switch is thought to be important in the regulation of natural sleep. Studies have been conducted to determine whether the VLPO nucleus is involved in mechanisms of anesthesia. In a study using rats as subjects, isoflurane increased activity in VLPO neurons but only one third as much as natural sleep. The membrane potential of cortical or thalamic neurons (i.e. level of hyperpolarization or depolarization) is the result of the sum of the directly acting hyperpolarizing effects of anesthetic agents and the depolarizing effects of the brainstem arousal system. If additional anesthetic agents such as opioids or dexmedetomidine could reduce the influence of the brainstem arousal system to the levels similar to that which occur in natural sleep, less anesthetic agent would be required to achieve the same degree of hyperpolarization of the thalamus and cortex.

If the spindle mechanism was in effect during anesthesia with very low activity of the brainstem arousal system, alpha oscillations would continue at low levels of anesthesia during emergence without much increase in frequency of the oscillation. If the brainstem arousal system was active during emergence the spindle mechanism would stop as the anesthetic agent level was reduced because the active brainstem arousal system would depolarize the thalamus and cortex. In that case the traveling peak mechanism could function and if it did the frequency of the alpha oscillation would increase well into the beta range unless the patient regained consciousness first.

The VLPO inhibits nuclei that project arousal neuromodulators. If the VLPO is “off” that only takes the “brakes” off. That does not cause the arousal nuclei to become fully active. Presumably, some stimulation must occur to cause the arousal system to become highly active. There is evidence that at reduced levels of anesthesia, activating the brainstem arousal system can result in emergence from anesthesia and inhibiting the arousal system delays emergence. If the brainstem arousal system activity was as low during anesthesia as it is during natural sleep and remained at that low level while anesthetic agents were eliminated, the thalamus and cortex would continue to be hyperpolarized despite anesthetic agent levels that were below the level where loss of response occurred during induction. At these low levels of anesthetic agent some effects from the agent would remain, but the effects related to low arousal system activity would become the dominant effect. In this scenario a subject could transition from anesthesia to a state indistinguishable from non-REM natural sleep.

The phenomenon of the alpha peak decreasing in amplitude without increasing in frequency seemed to occur most often when intravenous opioid was administered at levels high enough to result in very low respiratory rates or the absence of spontaneous respiration. These examples would be consistent with the spindle mechanism and the possibility of the brainstem arousal system being suppressed by opioid effects during emergence. If this phenomenon was due only to blocking of residual nociception from surgery, epidural anesthesia would produce the same effect. However, the cases with supplemental epidural anesthesia frequently developed a beta peak during emergence.

The phenomenon of the alpha peak increasing in frequency during emergence appeared to occur most often with either epidural or lower doses of intravenous opioid. In many of those cases the alpha peak frequency never increased to above 15 or 16 Hertz. It is possible that observation was achieved with a variation of the spindle mechanism and did not become the beta oscillation that is part of the traveling peak mechanism.

In other examples which demonstrated a clear beta peak during emergence, it appeared possible that a beta peak formed separately from the alpha peak. There were also examples with alpha and beta peaks clearly occurring at the same time and behaving independently. If the beta peak occurred simultaneously with the alpha peak and ended when the alpha peak ended it may be that some form of “harmonic” mechanism occurred which may be mostly a single mechanism. Perhaps there are two separate mechanisms but they become synchronized at harmonic multiples. If the beta peak continued after the alpha peak ended then they may have been caused by separate mechanisms.

The appearance of a beta peak cannot be explained by the spindle mechanism. There have been many articles published in the neuroscience literature regarding thalamocortical oscillations related to sleep spindles using a wide variety of experimental methods. There are no examples of sleep spindle related oscillations that can explain the “traveling peak” observation. A GABA effect on a cortical based 40 Hz rhythm which is the basis of the traveling peak mechanism is possible but is not the only possible explanation for a beta oscillation. There are 40 Hz oscillations generated in thalamic nuclei and the same GABA effect could be at work there.

Determining a level of opioid that would suppress the brainstem arousal system could be used to develop a strategy for the administration of anesthesia that would produce an emergence that would greatly reduce or possibly eliminate an excitement phase or delirium. An example of such a strategy might be as follows:
1) Anesthesia is induced in the usual fashion.
2) Intravenous opioid and the hypnotic agent (propofol or inhalation halogenated agent) levels are raised to a high enough level to likely put the brainstem in the mode of reduced projection of arousal neuromodulators (VLPO highly active).
3) Then the hypnotic agent is gradually reduced. Analgesia sufficient to prevent arousal is maintained and watched closely to prevent a decline below the critical level. In some embodiments, this might be achieved by a continuous infusion of remifentanyl.
4) In some embodiments, dexmedetornidine would be useful because of its unique effects on the brainstem arousal system.
5) Maintenance level of the hypnotic agent would be such that if the brainstem arousal system switched on, the cortical and thalamic effects of the hypnotic agent would prevent awareness.
6) Monitoring the EEG spectra would be beneficial to verify that hypnotic and analgesic agent levels are appropriate. Near the end of the procedure the hypnotic agent level would be reduced further. After the last potentially painful element of the surgical procedure ends, the hypnotic agent should be discontinued.
7) No stimulation by the anesthesia provider should occur until the hypnotic agents cortical effect has diminished enough so that consciousness will return at brainstem arousal.

In summary, induction is designed to flip the brainstem arousal system to the “off” state. Then the analgesia level is closely followed to prevent flipping the brainstem arousal system to the “on” state until the end of the procedure. High levels of propofol or inhaled agent are not required to prevent consciousness once the brainstem switch is flipped to the “off” state. If patient responses to surgical stimulation result in movement, hypertension, or tachycardia and the EEG pattern does not show any change in the alpha band oscillations, these responses can be interpreted as spinal cord or autonomic nervous system reflexes and treated with agents specific to those reflexes rather than increasing the level of the inhaled anesthetic agent or propofol. If the thalamocortical oscillation theory is correct and brainstem arousal activity could be controlled during anesthesia maintenance and emergence, then employing this anesthesia strategy could prevent intra-operative awareness, the excitement phase, and delirium during emergence.

The assertion that this strategy for the administration of an anesthetic could prevent intra-operative awareness, the excitement phase, and delirium during emergence relies on a thalamocortical oscillation based theory of anesthesia and consciousness as opposed to the continuous incremental impairment theory of anesthesia. The thalamocortical oscillation based theory relies on synchronized oscillations linking widespread areas of the cortex. During consciousness this is accomplished by means of 40 Hz oscillations. During non-REM sleep, spindle oscillations synchronize large cortical areas with 7-15 Hz oscillations in a manner that is not compatible with consciousness. The theory is that achieving and maintaining the anesthetized state involves a similar mechanism. The anesthetized state is not a lower “level of consciousness” but in contrast, no consciousness because the neurons are interacting in a manner that may be similar to how they interact during non-REM sleep and is considered to be incompatible with generating consciousness. The mechanism that produces switching between the two states is the change in membrane potential of thalamic neurons. Natural sleep and awareness switch back and forth due to brainstem activity changing the membrane potential of thalamic neurons. According to the theory, if an anesthetic regimen maintained the thalamus at a hyperpolarized membrane potential, then intra-operative awareness would be prevented. If an anesthetic regimen could reduce brainstem arousal activity to a level similar to non-REM sleep and maintain that activity level while the inhaled agent or propofol levels were reduced to low enough levels, the subject would return to consciousness when brainstem activity increases to levels typical of consciousness. Excitement and delirium would occur only if the brainstem arousal system was active but unable to overcome the hyperpolarizing effects of residual anesthetic agents and reestablish consciousness.

In order to implement the clinical strategy outlined above it is beneficial to determine the minimum level of opioid analgesia necessary as the level of inhaled agent or propofol is lowered. This can be done by using a series of EEG spectra and other data.

A common sequence in the practice of anesthesia is to induce anesthesia with enough quantity of anesthetic drugs for the patient to tolerate the high initial stress of endotracheal intubation and surgical incision. This is frequently a high enough level to induce the EEG pattern of burst suppression. It is common practice to reduce the level of anesthesia after incision. Maintenance of anesthesia is often below the level where burst suppression occurs as it is not usually necessary to maintain this high a level of anesthesia for the duration of the procedure. If the level of anesthesia is reduced too much the patient may react to the surgical manipulations with movement or cardiovascular reflexes. It is possible to safely reduce the level of anesthesia into that range and prevent such responses if enough analgesia is added to the anesthetic protocol.

It would be advantageous to have a method to analyze the EEG which would enable the anesthesia provider to reduce the level of agent without potentially harmful reflex responses to surgical stimulation.

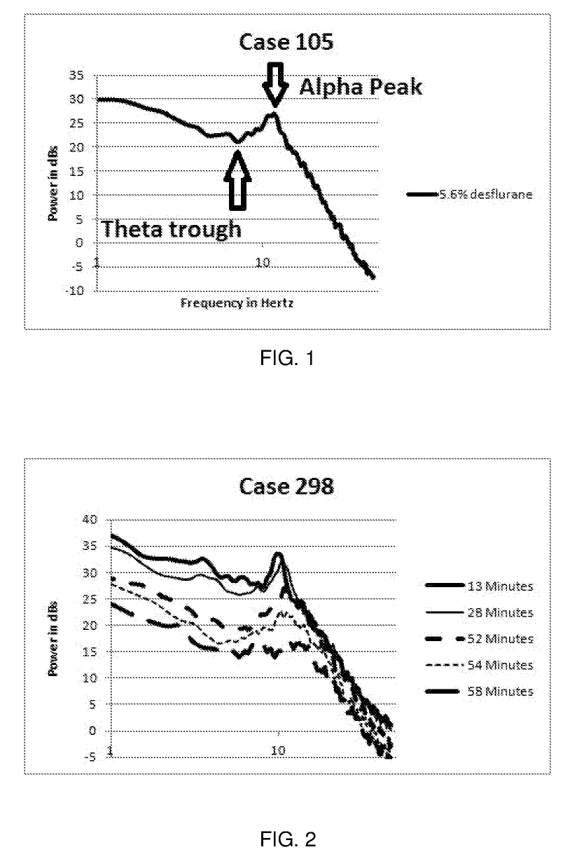

The EEG signal during anesthesia consists of multiple fluctuations in amplitude that appear to be the combination of multiple oscillations over a range of frequencies. The power (amplitude squared) at each frequency of the EEG signal can be determined with the fast Fourier method and displayed as a spectra (FIG. 1). The relative powers of different frequencies create an “EEG spectral shape”. The shape often consists of peaks, troughs between peaks, and if a log-log presentation is used there are often straight lines. As the anesthetic agent is reduced from a high concentration the power of each frequency will change. When the power at all frequencies change by the same amount the shape will not change but the overall power will. When the power of some frequencies changes more than others the shape will change. In many cases both the shape and the overall power will change. (That is, the power at some frequency ranges will change more than others.) EEG spectra can be recorded and displayed in an overlaid arrangement as the anesthetic agent is reduced from a high concentration. The series of changes in the spectra can be referred to as a “pathway” for that individual. FIG. 2 is a set of spectra from a patient which is an example of a pathway. The highest concentration of anesthetic agent occurs at the beginning of the case and decreases as time goes forward.

A group of similar patients receiving similar anesthetic regimens and producing the same pathway of EEG spectra as the anesthetic is reduced can be used as a reference for a new individual who appears to follow the initial part of the pathway. Since the sensitivity to anesthetic agents varies, an individual's EEG spectra within a series may be a better indication of that individual's level of anesthetic effect than a measurement of the concentration of the agent.

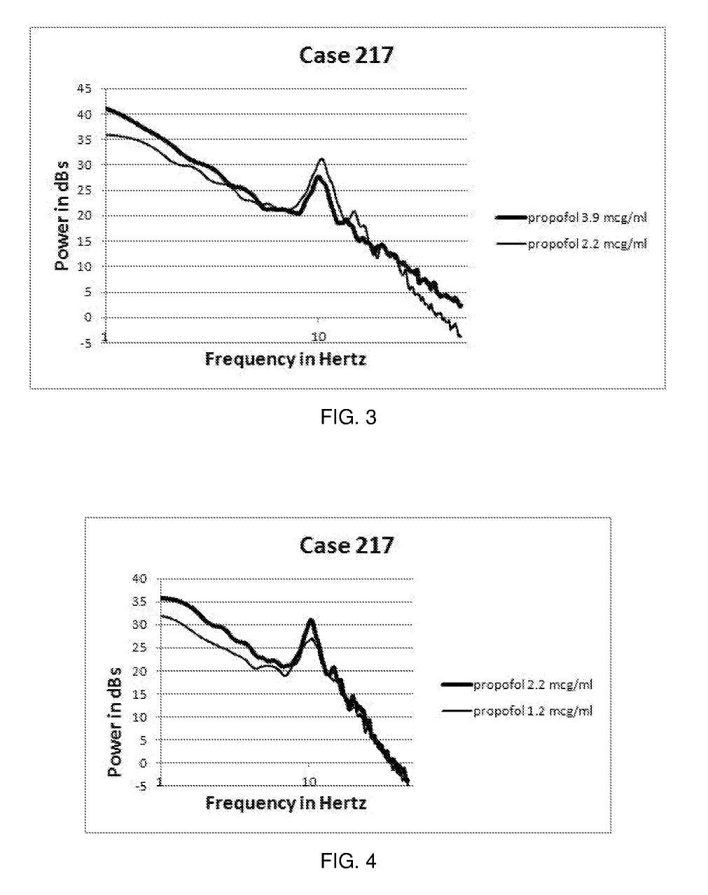

Propofol plus an opioid such as fentanyl is one example of a category of anesthesia protocol known as “total IV anesthesia” (TIVA). The change in EEG spectra shape and amplitude from high concentration to low concentration is biphasic. The initial change in the power of the alpha peak is to increase as the propofol concentration declines. Further reduction in the concentration results in the power of the alpha peak declining. The EEG spectra in the sequence with the maximum alpha peak power can be used for a reference spectrum in the pathway. There are other changes in the shape and amplitude of the EEG spectra during this process. The sections of the spectra above and below the alpha peak often form a straight line on a log-log presentation. These two segments can be referred to as the “high frequency segment” and the “low frequency segment”. EEG spectra from an anesthetic agent concentration above the maximum alpha peak power reference spectrum have a low frequency segment that is steeper than the low frequency segment of the reference spectrum and a high frequency segment that is less steep than the corresponding segment of the reference spectrum. These changes are illustrated in FIG. 3.

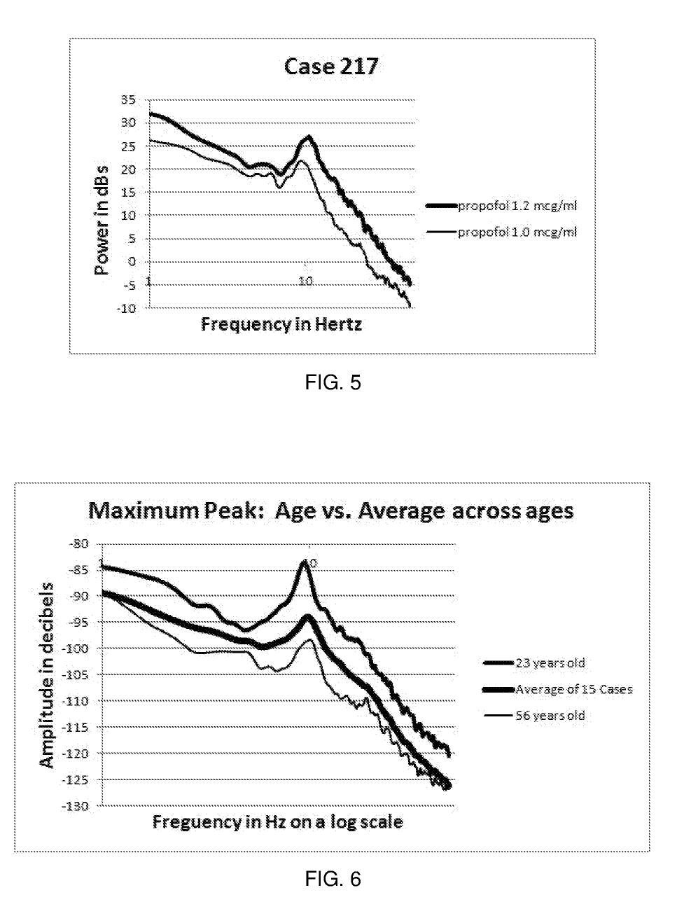

As the concentration of propofol is reduced further, the power at every frequency point from below and including the alpha peak is reduced. This is illustrated in FIG. 4. Further reductions can decrease the power at every frequency point of the spectrum (FIG. 5).

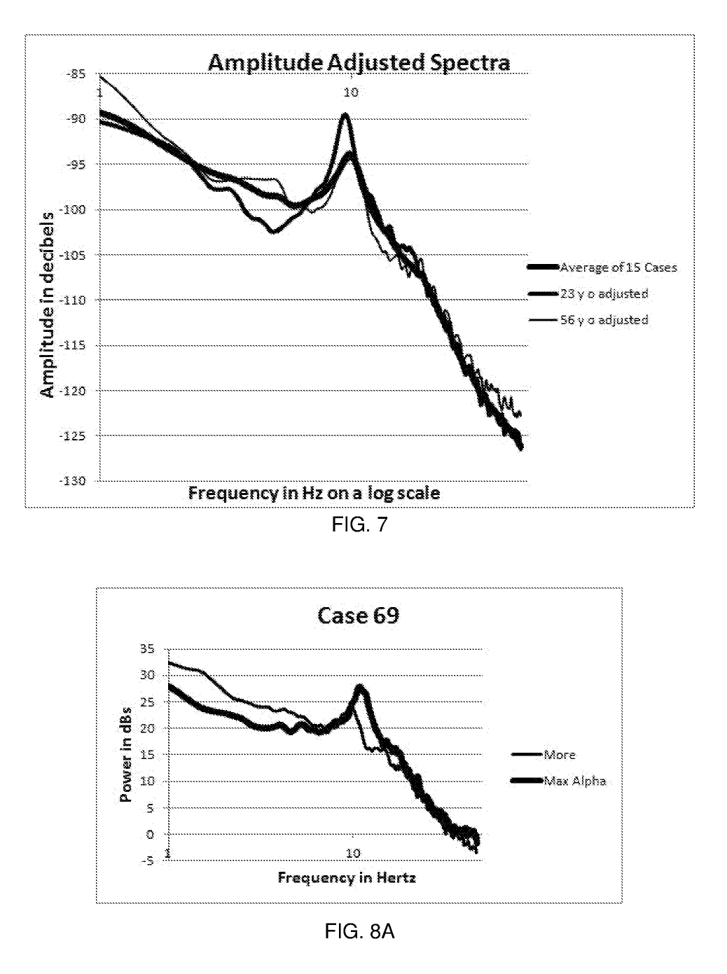

The power of EEG spectra at any point in the pathway is generally related to the patients age (FIG. 6). In order to use a set of spectra in a reference pathway to compare with the spectra of a patient it is beneficial to adjust the power in one of the two sets of spectra. This can be done by finding a spectrum in the patient that matches the maximum alpha peak spectra of the reference pathway and adjust the patient spectrum's power until the part of the two spectra at frequencies above the alpha peaks overlap. This is illustrated in FIG. 7, The amount of power adjustment is beneficially applied to all the spectra in the pathway.

The phase where the power of the entire EEG spectrum decreases can be used as an indication of the level of propofol if it is used as part of a sequence or pathway. A single spectrum in that range can have a shape similar to a spectrum at a higher or lower level of propofol, but without knowing the power adjustment needed for this patient, interpreting the patient's spectra can be difficult to perform accurately. This is in direct contrast to EEG based anesthesia monitors that rely on the current EEG spectral data without using it in the context of a pathway.

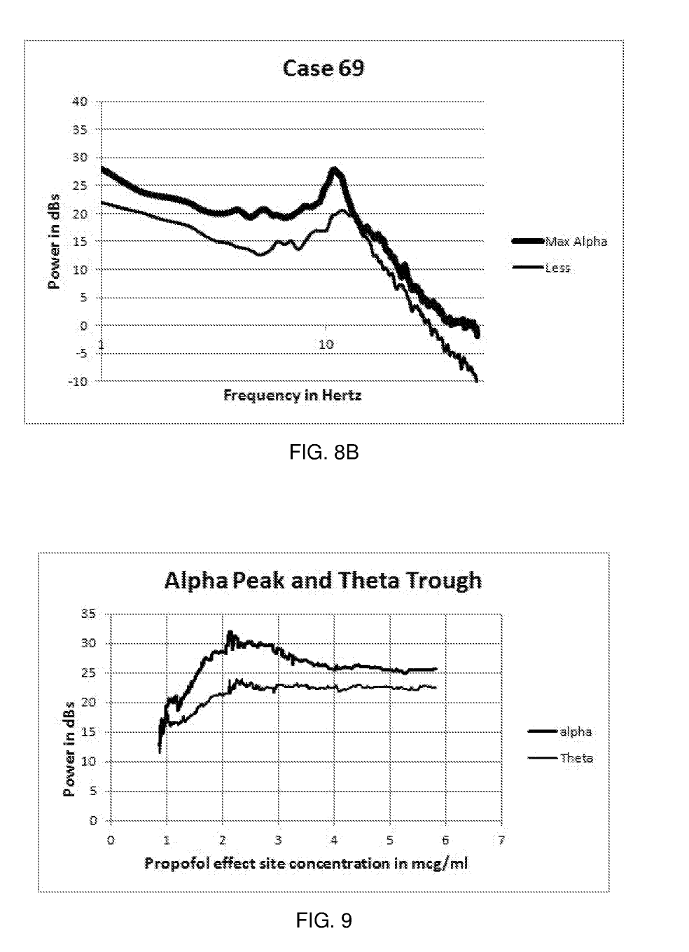

The maximum alpha peak spectral shape in the propofol plus opioid pathway often matches the maximum alpha peak spectral shape in the halogenated inhaled agent plus opioid pathway. The two pathways tend to diverge above and below that point in their pathways. The center frequency of the alpha peak usually shifts to lower frequencies and the power in the theta region (3.5-7 Hz) rises as the concentration of the inhaled agent increases above the concentration at the maximum alpha peak spectral shape. Below the concentration of the maximum alpha peak the opposite occurs. This is illustrated in FIGS. 8A-B.

Once it is determined that the subject with either TVA or inhaled halogenated agent anesthesia has spectra that match one of the known pathways it is possible to use a few parameters to track the pathway without matching the spectra. The most useful parameters are the power of the center frequency of the alpha peak, the power of the theta trough or peak, the frequency of the alpha peak and the slope of the high frequency approximation line. This is illustrated in FIG. 9. One problem with this method is that if the subject's spectral shapes no longer match the spectra in the reference pathway the parameters alone may no longer be an accurate representation of the subjects level of anesthesia.

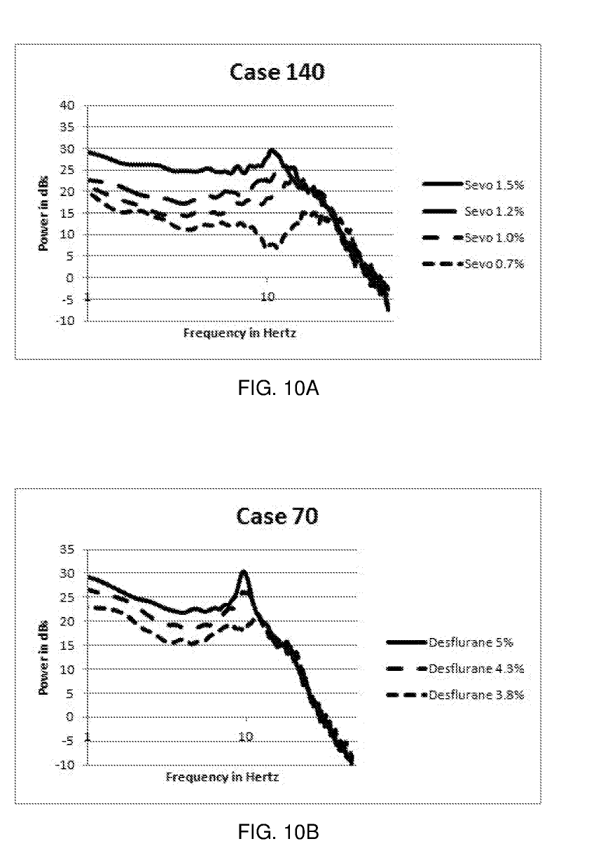

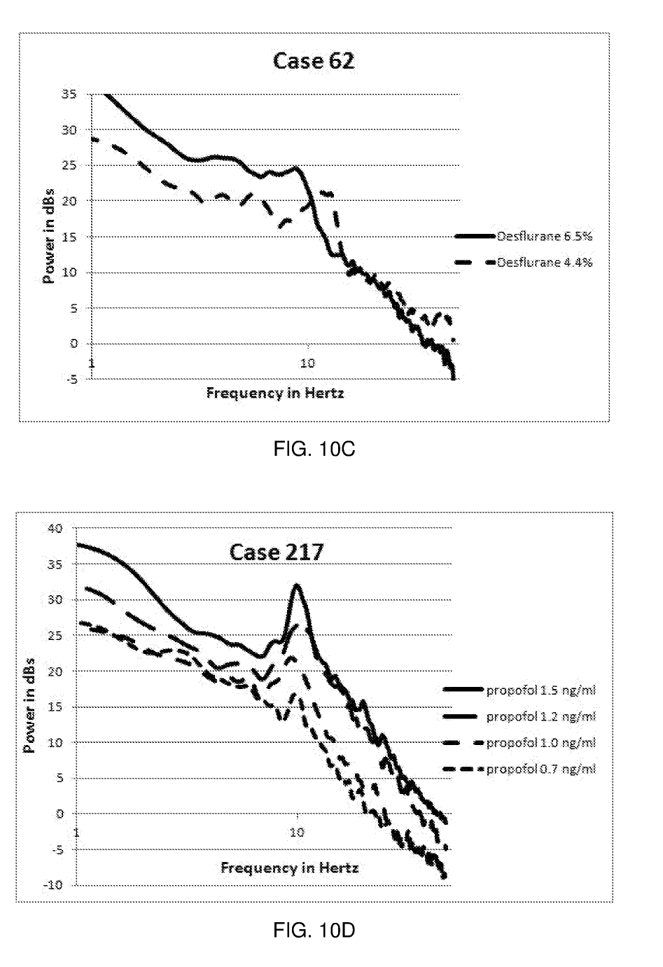

Evaluating pathways from many individuals reveals that there are several pathways that occur. FIG. 2 and FIGS. 10A-D show several pathways (e.g. 5 different pathways). This implies that utilizing EEG spectral data to determine the probability of a reflex response to surgery for an individual should first determine which EEG pattern pathway to use. A universal quantitative EEG algorithm based on spectral analysis as used in several commercial EEG “depth of anesthesia” monitors is inaccurate for many individuals for this reason. In accordance with the principles of the present disclosure, accuracy for an individual is improved by utilizing a method that both identifies the correct pathway and the present location on the correct pathway. Patient factors and choice of anesthesia technique determine which pathway occurs.

Embodiments in accordance with the present invention capture EEG signals for a patient during initial stages of being administered anesthesia. During the initial stages, the anesthesia typically includes a relatively higher level of primary agent and a relatively lower level of analgesic agent. Historical data related to the anesthesia being administered to the patient is also available. The patient's EEG signals are used to search the historical EEG signals to identify an EEG signal similar to the patient's EEG signal. The similar EEG signal is selected as a reference pathway comprised of a number of “steps” wherein each “step” represents a respective EEG signal as it changed during a procedure. In reality, the EEG signals of the reference pathway can be an average of EEG signals from multiple subjects. The reference pathway represents the progression of the EEG signals that did not result in a subject having a response to surgical stimulation. As the primary agent and analgesic agent are changed during a surgical procedure, a determination is made whether the patient's current EEG is similar to the next “step” (i.e., EEG signals) in the reference pathway. The expectation is that the patient will likely respond in a manner similar to that of the reference pathway and, thus, at each step the patient's EEG is likely to be similar to the EEG of the corresponding step of the reference pathway. Displaying the two EEG signals in a manner that provides evidence that they are similar to one another provides a clinician when reassurance that the combination of primary agent and analgesic agent being administered to the patient is sufficient to prevent the patient from responding to surgical stimulation.

In some instances the patient's EEG signals may closely match those of the reference pathway but the combination of anesthetic agent and analgesic agent may not be sufficient to prevent a response. Thus, physiological responses to surgical stimulation can be monitored as well. If a patient's heart rate rises more than a predetermined threshold (e.g., 10%) or the patient moves, then this information can also be used to determine that the patient is not following an expected course of behavior.

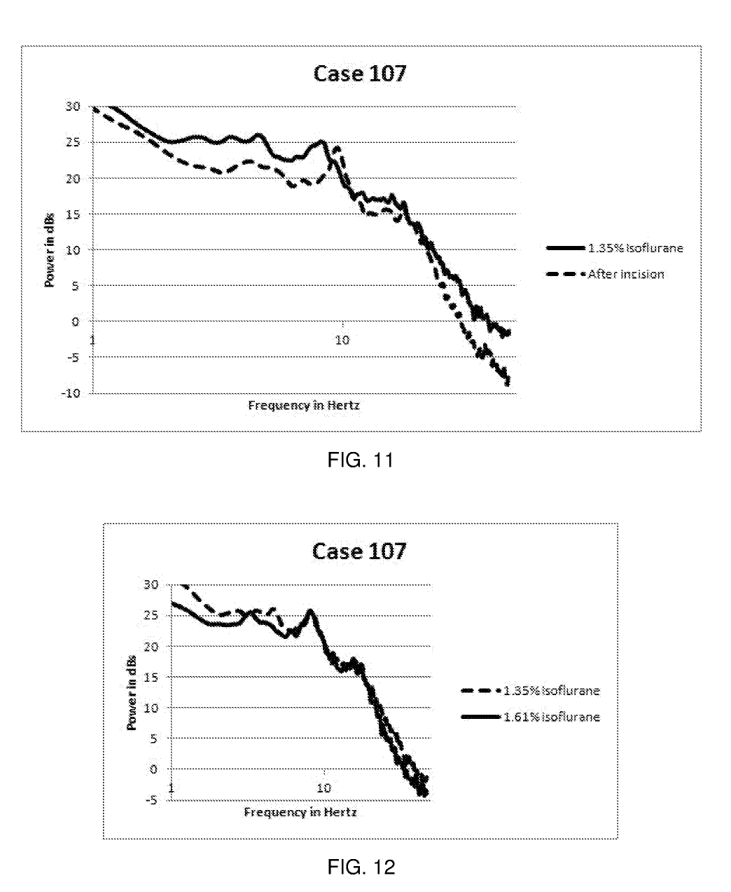

Surgical stimulation can change the shape and amplitude of the EEG spectral pattern. FIG. 11 shows subject in at 1.35% isoflurane before and after incision. The effect of increasing the primary anesthetic, agent without adding an analgesic agent is illustrated in FIG. 12. This is the same subject and demonstrates that it took a higher level of isoflurane to produce a similar EEG spectral pattern during surgery than it did before incision.

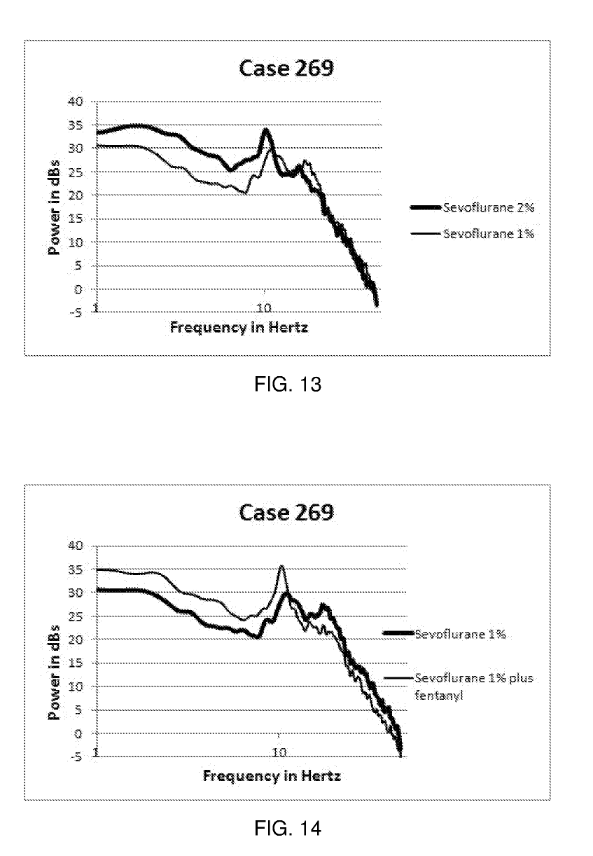

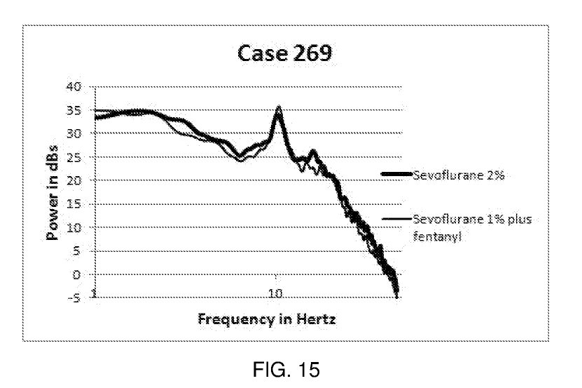

Increasing the anesthetic agent or adding analgesia with opiate, regional anesthesia or other types of agent can partially or completely reverse the change due to stimulation. FIG. 13 demonstrates a major change in the EEG spectra after sevoflurane is reduced from 2% to 1% during surgery. Fentanyl was administered and fentanyl partially reversed the changes that occurred after the primary agent sevoflurane was reduced as illustrated in FIG. 14. FIG. 15 compares the spectra at 2% sevoflurane with the spectra at 1% sevoflurane and a higher level of fentanyl.

FIGS. 13-15 demonstrate that for a model of anesthetic requirement to utilize the EEG spectral pattern, the model, in accordance with the principles of the present disclosure, interprets the EEG spectral pattern in the context of surgical stimulation and the level of analgesia.

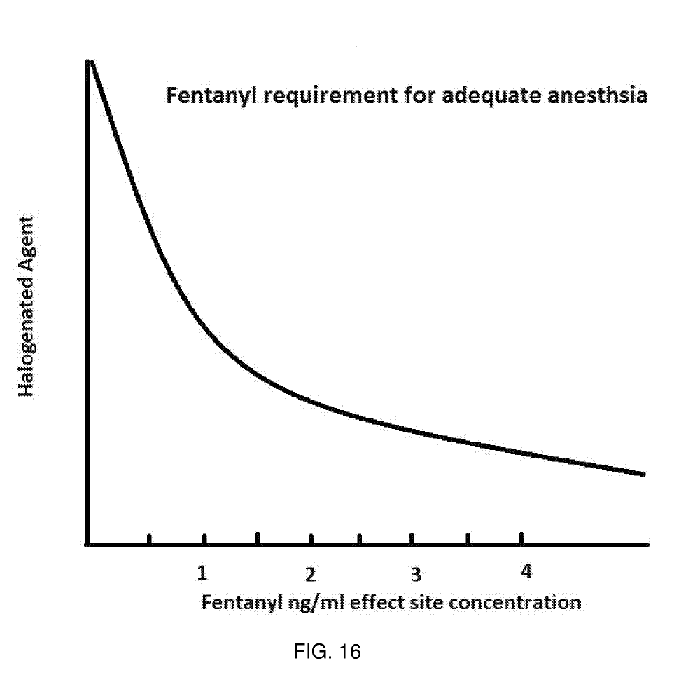

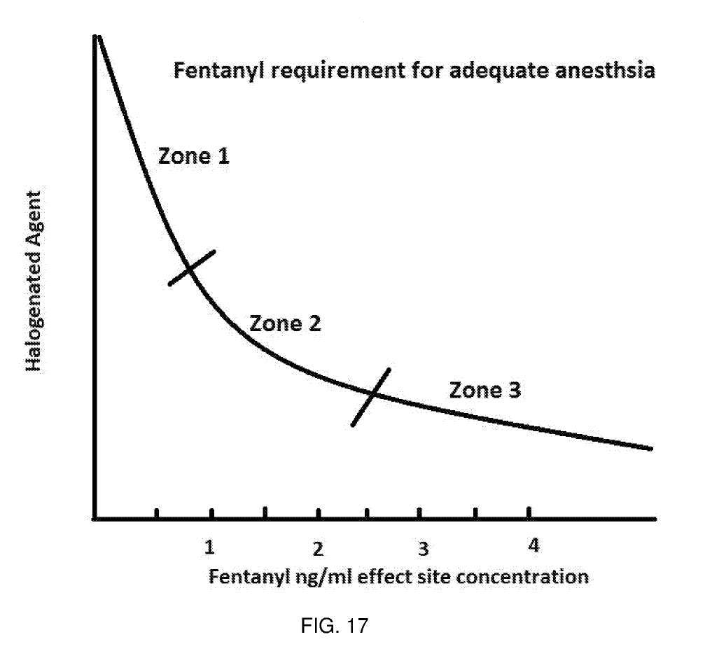

FIG. 16 is a diagram that demonstrates the relationship between the effective concentrations for a halogenated agent and an opiate analgesic agent for adequate anesthesia in an “average” subject. The curve showing the relationship between hypnotic and opioid requirement has three zones (FIG. 17). In zone one a small amount of opioid is all that is required to prevent a response to surgery. In zone two additional reductions in hypnotic agent require increasing amounts of opioid. In zone three small reductions in hypnotic agent require increasingly high levels of opioid. Zone two is the most efficient zone. The other zones can be seen as unbalanced with an excessive amount of one of the two agents.

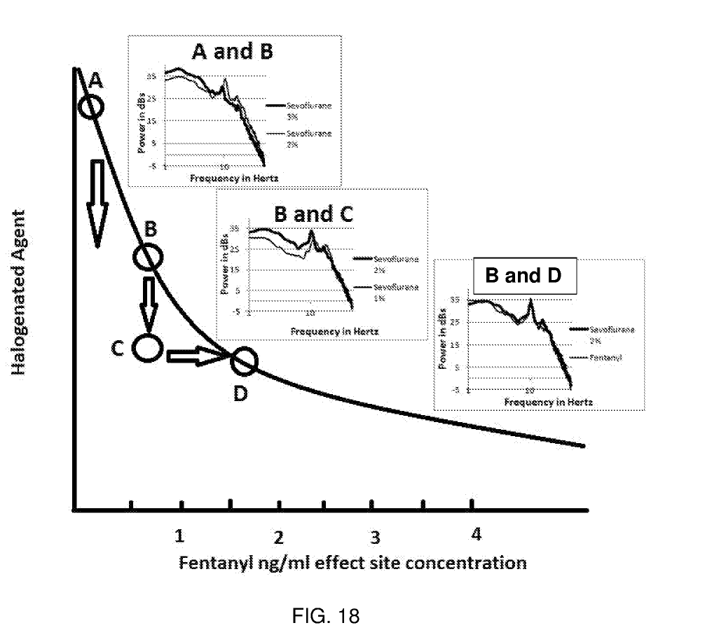

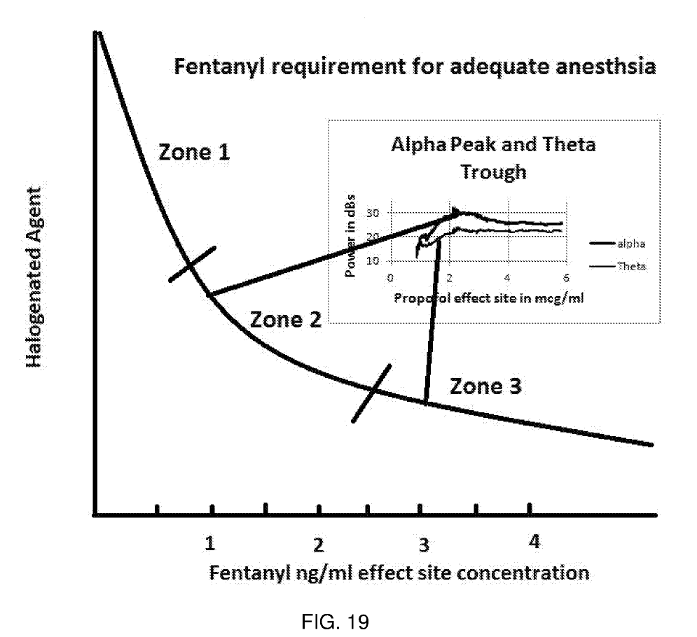

The EEG spectra path can be used to place a subject on the curve in FIG. 18. The EEG spectra in a pathway are useful for this purpose. It is possible to use the parameters extracted from the spectra as illustrated in FIG. 19 for this purpose. Zone two is where the maximum alpha peak occurs. Zone three is where small reductions in hypnotic agent produce large reductions in the power of the alpha peak and the theta trough or peak. Zone three is where reductions in the opioid effective concentrations result in large changes in the spectra which can be reversed by additional opioid. Thus, FIGS. 16-18 illustrate how a change in an EEG signal can be used to maintain adequate anesthesia as the primary agent is decreased by having a reasonable estimate of the amount of opioid analgesic required with each reduction of the primary agent. In FIG. 18, the change in primary agent from B to C. (e.g., 2% to 1%) results in the changed EEG power spectrograms that include a change in alpha peak frequency and the presence of a beta peak. The addition of fentanyl (to the 1% sevoflurane) in an amount to achieve position D results in the EEG signals that are very similar to one another even though the primary agent has been reduced from 2% to 1%.

The line of FIG. 18 represents a two-dimensional graph of the probability that a patient, based on a population of subjects physiologically similar to the patient, will respond to surgical stimulus. Values for the combination of primary agent and analgesic agent that are above the line represent a probability that the patient will not respond while values below the line represent a probability that the patient will respond. A “response” can include, for example, a 50% likelihood that the patient will move, a 95% chance that the patient's heart rate will increase by a particular amount, or a 10% increase in a patient's blood pressure.

Regardless, historical data can be collected and analyzed to determine the shape, slope, and other characteristics of the line of FIGS. 16-19 and associated with different EEG signals such as the power spectrograms shown in FIG. 18 and the EEG spectrum data shown in FIG. 19. Thus, as a level of the primary agent is lowered (e.g., from B to C) an estimate of the amount to increase the analgesic agent can be determined so as to remain on (or within a predetermined proximity) to the line of FIGS. 16-19.

In the particular EEG signals of FIG. 18, the EEG signal at position C with the smaller amplitude power at low frequencies, the lower and shifted alpha peak, and the presence of a beta peak is indicative of a state of anesthesia wherein the patient is more likely to respond to surgical stimulus than a subject having an EEG signal similar to that at position B. The line of FIG. 18 provides an estimate of how much additional analgesic agent is likely to cause the EEG signal of the patient to return to being similar to that at position B. Thus, the curve or graph of FIG. 18 in conjunction with identifying matching EEG signals can be used to identify a combination of anesthetic agent and analgesic agent which will achieve a desired level of anesthesia to minimize the patient's likelihood of responding to surgical stimuli. At position B, the patient has a first probability of responding to surgical stimulation but at position C, the patient has a different, second probability of responding to stimulation (e.g., more likely to respond). An accompanying change in the patient's EEG signal or other physical responses can confirm the greater likelihood of responding. The horizontal distance from position C to the line or graph of FIG. 18 provides an estimate of the amount to increase an analgesic agent in order to return to substantially the first probability. The term “substantially” recognizes that the return to the first probability may not be exact and includes a return to a probability that is within a predetermined proximity of the first probability (e.g., 1%, or 5%, or 10%, etc.).

It is not to be inferred that any system of parameters or indices based on, extracted from, or created from EEG data, spectral or otherwise related to the EEG signal will work as well for this purpose as the EEG spectral pathway concept.

What follows is an example of how to use a series of EEG spectra to optimize an anesthetic.
1) For induction and before the start of the surgical procedure one would adjust the propofol or inhaled agent to a level above the maximum alpha peak. A skilled artisan would know an estimate of this level based on the primary agent and physiological attributes of the patient.
2) Begin reducing the level of propofol or inhaled agent while maintaining a low level of analgesia with about one nanogram/milliliter of estimated fentanyl effect site concentration.
3) As the level of the propofol or inhaled agent is reduced there will be a gradual change in the patients spectra until that level is below the level of propofol or inhaled agent that resulted in the maximum alpha peak power in the selected reference pathway of spectra. The level of anesthetic agent at the maximum alpha peak is actually a zone and not a single value.
4) Then there will be larger declines especially in the alpha and theta regions with small reductions of the anesthetic agent. There may be the appearance of a beta peak or a shift of the frequency of the alpha peak into the beta range. At this level of anesthesia the opiate will affect the spectra by counteracting the effect of surgical stimulation as illustrated in FIGS. 13-15. At this level of halogenated agent or propofol a high dose of opiate may alter the EEG spectra by direct action.
5) The anesthesia provider can decide to use this low level of propofol or halogenated agent with higher levels of opiate analgesia or to go back up with the propofol or halogenated agent and use a lower level of opiate anesthesia. In either case, the goal is to control the level of the agent and/or opiate to cause the patient's current EEG spectrum to be similar to the appropriate spectrum from the selected reference pathway.
6) Another option is to start the anesthetic as total IV anesthesia and if the EEG spectra changes indicate that the surgical stimulation is not effectively blocked by moderate concentrations of propofol and opiate, a small amount of halogenated agent will be added.

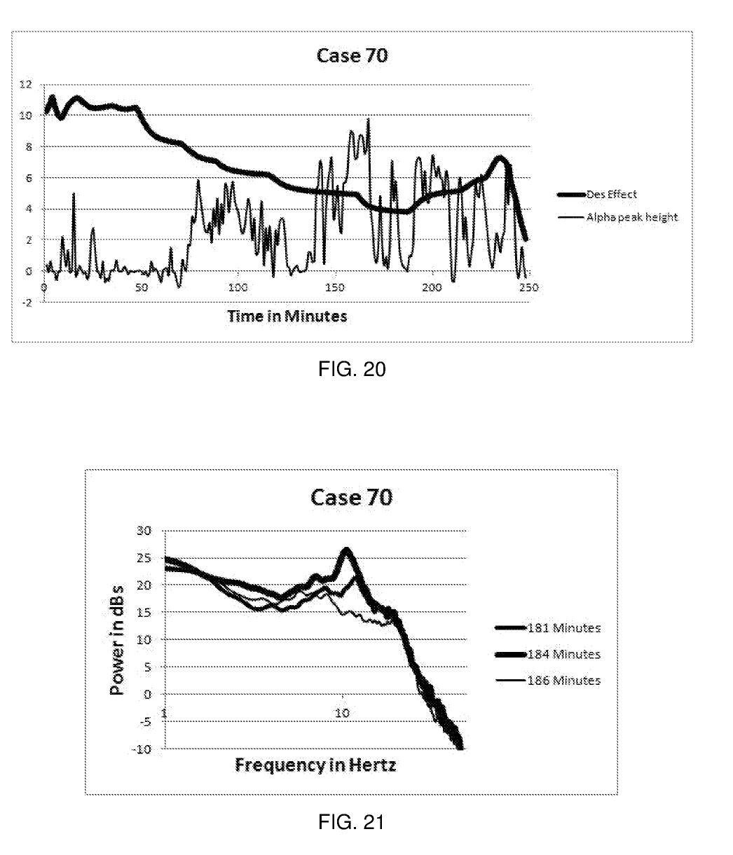

If the anesthetic agent level is sufficiently high, the response to surgical stimulation will be largely a cardiovascular reflex without a movement response. If this occurs for several minutes, the cardiovascular reflex will likely decrease. This is indicated by an initial increase and then a decrease in the heart rate. If the anesthetic agent is then reduced repeatedly in stages without adding an analgesic agent, it may be possible for the patient to tolerate surgical stimulation at a level of anesthetic agent so low that the patient would be expected to move in response to incision. This is explained by a neural process termed “accommodation”. This sequence is illustrated in FIG. 20. At this low level of anesthetic agent the patient did not move but had changes in the EEG spectra (FIG. 21). The changes in the EEG spectra that are observed in the above example mimic lower levels of inhaled agent.

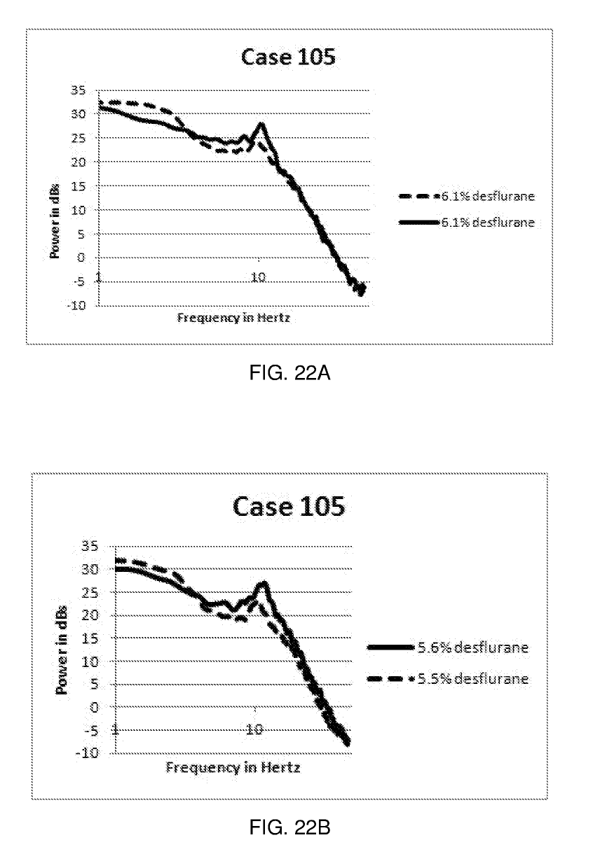

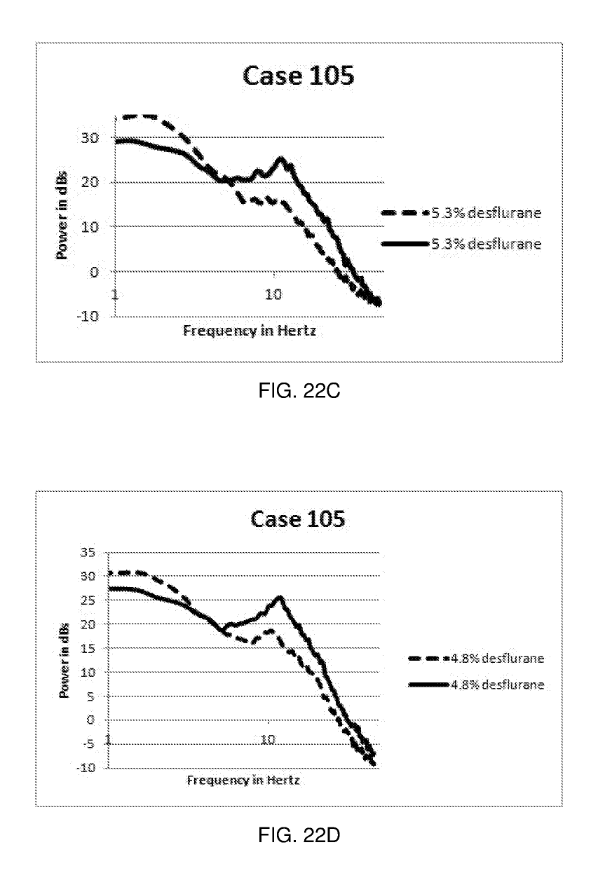

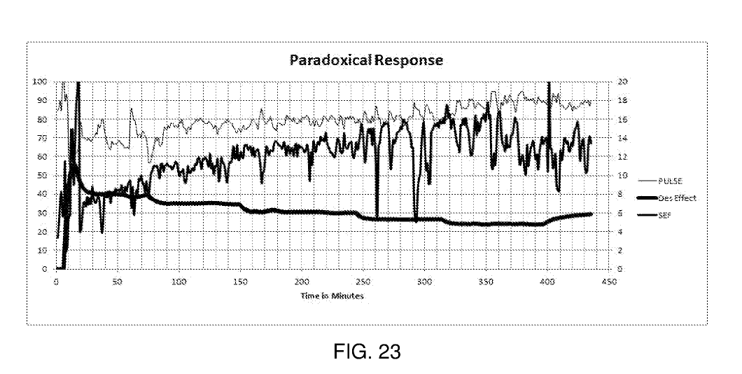

Another pattern can also occur which is termed the “paradoxical response”. In that case the EEG spectral changes mimic a higher level of inhaled anesthetic agent. FIGS. 22A-D show a series of paradoxical responses at different agent concentrations. It is possible that the paradoxical response is due to a subject activating their intrinsic analgesic responses. This could be used to administer anesthesia without opiate agents. One would lower the inhaled agent until the paradoxical response is maximized and maintain that concentration. In the case illustrate in FIGS. 22 A-D the concentration of maximum paradoxical response was 5.3%.

The response could be tracked with a diagram as illustrated by FIG. 23. This illustrates another way to use EEG spectral pathways to provide useful information to the clinician administering anesthesia. The method can be applied to not using opiate analgesics or using other agents in an anesthetic protocol such as ketamine, lidocaine, or other agents.

If the patient has an effective level of analgesia due to the administration of an opiate (such as fentanyl to achieve an effect site level in the range of about 1-4 ng/ml) there will likely be very little cardiovascular responses to the surgical stimulation as the primary anesthetic agent is reduced. If the primary agent is reduced, the anesthetist should maintain and probably increase the level of analgesic agent since the presence of analgesia has prevented the phenomenon of accommodation. It is also to be remembered that accommodation is often specific to the area of the body where surgery is performed. For instance, if the surgeon is going to operate on both legs, the development of accommodation on one leg will not prevent a response when surgery starts on the other leg.

The alpha peak and the theta trough or peak have been discussed. The beta peak or “hump” (plateau) can also be useful. FIG. 2 shows an example of the opiate plus inhaled agent pathway. There is no bump or peak in the beta frequency range (14-25 Hertz) in FIG. 2. FIG. 11 shows two spectra with a beta “hump” or plateau. There was no opiate used in this case. FIGS. 13 shows a beta hump becoming a beta peak when the inhaled agent is reduced. FIG. 14 shows that the beta peak is reduced back to a plateau with the addition of fentanyl without changing the level of the inhaled agent. These are examples of how a specific feature in EEG spectra within a pathway can be used to determine the effects of the level of opiate analgesia and the effectiveness of increasing the level of opiate analgesia.

An example method for implementing the above described embodiments can include the steps of:
1) Record and digitize a patient EEG signal.
2) Transform EEG to a log-log power spectrogram as described in the above-identified patents and patent applications incorporated by reference.
3) Use a pattern match algorithm, as described in the above-identified patents and patent applications incorporated by reference, to find all possible matches for individual spectra and hence all possible pathways.
4) Use series of spectra and other data (age, measurements provided by other monitors and operator input) to identify pathway and location on pathway.
5) Adjust the power of one of the spectra to produce a match with an appropriate spectrum in the reference pathway. Use the amount of the adjustment for subsequent spectra.
6) Locate current situation of patient for opiate recommendation for this point in the pathway. For example, the anesthetic agent can be reduced as compared to the pathway and the analgesic agent increased so that the current EEG spectra or data continues to be similar to the EEG spectra or data of the pathway.
7) Detect indications of response to surgical stimulation during the entire recording on this patient. The response can be detected based on the patient's current EEG data deviating from the expected EEG data represented in the pathway.
8) Use responses to surgical stimulation to modify opiate level versus response probability table. The responses can include a patient's increased heart rate, increased blood pressure or movement. The response can also include a change in the EEG spectra or data.
9) Display information for anesthesia provider. The displayed information can be overlaid spectrograms of the patient and the pathway or can be other information indicative of a similarity between the patient's EEG signal and the EEG signals of the pathway,

Example embodiments have been described above that rely on power spectrograms of EEG signals to be utilized in determining deviations of a patient's behavior from an expected pathway. However, one of ordinary skill recognize that other presentations, derivations, and methods to characterize or parameratize the EEG signal can be used without departing from the scope of the present disclosure. The underlying shape and characteristics of an EEG signal is based on oscillations that are detected within different frequency ranges. Thus, various methods of characterizing an EEG signal in a way that captures the attributes of the different oscillations can be used in order to compare a patient's current EEG signal with that of an expected EEG signal on a reference pathway. As described above, for example, the slope of a high-frequency line in a log-log power spectrogram can be compared, the frequency and amplitude of an alpha peak can be compared, the differences in the beta-frequency ranges can be compared, the power amplitude in different frequency regions can be compared, the absence of a peak can be determined, or the theta trough or peak regions can be compared. Thus, features are the result of oscillations in the networks of neurons in the brain that generate the signal are within the scope of what can be compared to determine the differences in EEG signals described above.

A pathway is a series of characterizations of the EEG signal as the effective concentration of the primary anesthetic agent (“hypnotic” effect agent, i.e. inhalational anesthetic or intravenous anesthetic (propofol, etomidate, barbiturate, etc.)) changes over a range of values. Thus, a pathway captures such information as changes in the series of spectral (or other characteristics) of the EEG signal that can occur because of changes in the primary agent, stimulation (i.e. surgical or other manipulations), other agents being added such as opiate analgesics (but not limited to) the effect of added agents or changes in the primary agent on the responses. The changes can also include any characteristic of the EEG signal plus any additional physiological signal (i,e. cardiovascular, respiratory, autonomic nervous system, etc.)

In general, the characteristics of the patient pathway are compared to data from a library of pathways to provide information to an observer that indicates what happened to others with a similar pathway is likely to happen to the current subject. Thus, an indication can be provided about the probability and nature of responses to stimulation at current levels of agents as well as changes in the probability if agent levels are changed. Furthermore, the patient continues to be monitored in order to determine the probability changes with new data from the subject such as responses that occurred.

Evaluating Electroencephalogram (EEG) data includes receiving initial EEG signals for a patient being subjected to an anesthetic agent to minimize a response to surgical stimulation; based on a similarity between the initial EEG signals and a corpus of historical EEG signals, identifying a reference series of EEG signals, wherein the reference series of EEG signals comprises a time-ordered sequence of expected EEG signals for the patient; receiving a subsequent EEG signal for the patient after a change to a level of the anesthetic agent has occurred; and displaying a difference between the subsequent EEG signal and a one of the expected EEG signals, wherein the difference is indicative of a probability that the patient will respond to surgical stimulation.

Evaluating Electroencephalogram (EEG) data includes receiving initial EEG signals for a patient being subjected to an anesthetic agent associated with a first probability for responding to surgical stimulation; identifying at least one expected pathway of EEG signals, wherein the at least one expected pathway of EEG signals comprises a time-ordered series of expected EEG signals for the patient based on the anesthetic agent; receiving a subsequent EEG signal for the patient after a change to a level of the anesthetic agent has occurred; based on the change to the level of the anesthetic agent, determining a change from the first probability to a second probability for responding to surgical stimulation; and calculating an amount of increase to an analgesic agent to administer to the patient to return substantially to the first probability for responding to surgical stimulation.

The above described embodiments include tracking a series of EEG spectra from a subject as the subject continues to be under the influence of an anesthetic agent and/or an analgesic agent. Based on the tracking, matching the patient's series of EEG spectra to a series of EEG spectra from a collection of EEG spectral series can be performed based on their distribution of power over the entire EEG frequency range (i.e. their “shape”). As part of the matching, a relative weight can be assigned to each shape feature so that it is given more or less importance. For example, the power (amplitude) or frequency of the alpha peak can be given one weight while the theta trough or peak or the slope of a high-frequency region can each be assigned a respective different weight. Each EEG signal can be characterized based on a combination of weighted features to determine whether or not a match occurs. As used herein, a match does not imply absolute equality but, rather, implies a statistical calculation indicative of the two EEG signals being similar to one another within a predetermined threshold. The matching also can involve compensating for variations in EEG power between individuals. As a result, the spectra in a series (both the patient's and the reference) can be associated with an estimate of the effective concentration of the anesthetic and analgesic agents which allows adjusting the parameters of the method of estimating the effective concentration of the agents as indicated by changing (or not changing) spectra. Furthermore, the spectra in a series can be associated with indicators of physiology as pertains to the level of response to surgical stimulation for both the current subject and such indicators in the reference series such that the spectra in a series can be associated with indicators of the level and nature of surgical stimulation.

As described in the above-noted patents and patent applications incorporated by reference, the steps and processes of the above-described methods can be implemented on a computer-based platform so as to occur autonomously. The probability curves and graphs of FIGS. 16-18 can be stored so as to be accessible to computer software executing on a processor, The patient's EEG signals as well as the historical pathways EEG signals can be collected, analyzed, manipulated and stored using computer software executing on a processor. Thus, the similarity and/or differences between two EEG signals can be determined and characterized by the computer software, Furthermore, the computer platform can include a display which presents information to a user about the differences in EEG signals, As one example, the display can include two EEG signals (e.g., a pathway signal and a patient signal) overlaid on one another so that differences between the two are visually discernable. Thus, embodiments of the present invention include a processor in communication with a memory that stores executable instructions. The processor when executing the instructions performs the operations to achieve and implement the methods and functional steps described herein. to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean one and only one unless specifically so stated, but rather “one or more.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public, regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. A method for evaluating Electroencephalogram (EEG) data comprising:
receiving initial EEG signals for a patient being subjected to an anesthetic agent associated with a first probability for responding to surgical stimulation;
identifying at least one expected pathway of EEG signals, wherein the at least one expected pathway of EEG signals comprises a time-ordered series of expected EEG signals for the patient based on the anesthetic agent;
receiving a subsequent EEG signal for the patient after a change to a level of the anesthetic agent has occurred;
based on a difference between the subsequent EEG signal and one EEG signal from the at least one expected pathway of EEG signals, determining a change from the first probability to a second probability for responding to surgical stimulation; and
calculating an amount of increase to an analgesic agent to administer to the patient to return substantially to the first probability for responding to surgical stimulation.

2. The method of claim 1, comprising calculating a combination of the anesthetic agent and the analgesic agent to return substantially to the first probability.

3. The method of claim 1, wherein identifying the at least one expected pathway of EEG signals comprises:
based on a similarity between the initial EEG signals and a corpus of historical EEG signals, identifying the at least one expected pathway of EEG signals.

4. The method of claim 3, wherein the similarity comprises a similarity between respective high-frequency attributes of the initial EEG signals and the at least one expected pathway of EEG signals.

5. The method of claim 3, wherein the similarity comprises a similarity between a first attribute associated with a first alpha peak of the initial EEG signals and a second attribute associated with a second alpha peak of the at least one expected pathway of EEG signals.

6. The method of claim 5, wherein the first attribute comprises a first power value of the first alpha peak and the second attribute comprises a second power value of the second alpha peak.

7. The method of claim 5, wherein the first attribute comprises a first frequency value of the first alpha peak and the second attribute comprises a second frequency value of the second alpha peak.

8. The method of claim 3, wherein the similarity comprises a similarity between a first theta trough or peak of the initial EEG signals and a second theta trough or peak of the at least one expected pathway of EEG signals.

9. The method of claim 1, wherein a response to surgical stimulation comprises the patient moving.

10. The method of claim 1, wherein a response to surgical stimulation comprises an increase in the patient's heart rate above a predetermined threshold.

11. A system for evaluating Electroencephalogram (EEG) data, the system comprising:
a memory storing executable instructions; and
a processor in communication with the memory and configured, when executing the executable instructions to perform operations comprising:
receiving initial EEG signals for a patient being subjected to an anesthetic agent associated with a first probability for responding to surgical stimulation;
identifying at least one expected pathway of EEG signals, wherein the at least one expected pathway of EEG signals comprises a time-ordered series of expected EEG signals for the patient based on the anesthetic agent;
receiving a subsequent EEG signal for the patient after a change to a level of the anesthetic agent has occurred;
based on a difference between the subsequent EEG signal and one EEG signal from the at least one expected pathway of EEG signals, determining a change from the first probability to a second probability for responding to surgical stimulation; and
calculating an amount of increase to an analgesic agent to administer to the patient to return substantially to the first probability for responding to surgical stimulation.

12. The system of claim 11, the processor when executing the executable instruction is configured to perform operations comprising:
calculating a combination of the anesthetic agent and the analgesic agent to return substantially to the first probability.

13. The system of claim 11, wherein identifying the at least one expected pathway of EEG signals comprises:
based on a similarity between the initial EEG signals and a corpus of historical EEG signals, identifying the at least one expected pathway of EEG signals.

14. The system of claim 13, wherein the similarity comprises a similarity between respective high-frequency attributes of the initial EEG signals and the at least one expected pathway of EEG signals.

15. The system of claim 3, wherein the similarity comprises a similarity between a first attribute associated with a first alpha peak of the initial EEG signals and a second attribute associated with a second alpha peak of the at least one expected pathway of EEG signals.

16. The system of claim 15, wherein the first attribute comprises a first power value of the first alpha peak and the second attribute comprises a second power value of the second alpha peak.

17. The system of claim 15, wherein the first attribute comprises a first frequency value of the first alpha peak and the second attribute comprises a second frequency value of the second alpha peak.

18. The system of claim 13, wherein the similarity comprises a similarity between a first theta trough or peak of the initial EEG signals and a second theta trough or peak of the at least one expected pathway of EEG signals.

19. The system of claim 11, wherein a response to surgical stimulation comprises the patient moving.

20. The system of claim 11, wherein a response to surgical stimulation comprises an increase in the patient's heart rate above a predetermined threshold.
